(19) United States
(12) Patent Application Publication
(10) Pub. No.: US 2008/0140199 A1
(43) Pub. Date: Jun. 12, 2008

(54) Impantable Body for Spinal Fusion

(76) Inventor: Arne Briest, Karlsruhe (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/572,436

(22) PCT Filed: Jul. 16, 2005
(86) PCT No.: PCT/EP05/07769

§ 371 Date: Aug. 17, 2007

(30) Foreign Application Priority Data
Jul. 21, 2004 (DE) 10 2004 036 954.2

(51) Int. Cl. A61F 2/44

(52) U.S. Cl. 623/17.11

(57) ABSTRACT

An implantable body for intersomatic fusion (spinal fusion) is disclosed, made from a bioresorbable, metallic material. Said metallic material preferably contains magnesium or iron as main component. The material is particularly a magnesium alloy or an iron alloy.

The present invention relates to an implantable body for intersomatic fusion or spinal fusion.

Back pain is a major problem that affects considerable numbers of people. A common cause of back pain lies in defects or degeneration of the intervertebral disks. The intervertebral disks are arranged between the individual vertebrae of the spinal column and ensure the mobility of the individual vertebrae relative to one another. Damage to the intervertebral disks may occur as a result of degenerative changes, injuries, excessive strain or personal disposition and can lead to considerable pain.

In many cases, the only way of helping the patient is to perform a surgical intervention in which the affected intervertebral disk material is removed and the adjacent vertebrae are fused or stiffened. In doing so, it is necessary to suitably fill the space that is created between the vertebrae, in order to avoid a collapse of the vertebral bodies, as this would cause an instability of the spinal column, with various adverse consequences.

For this purpose, an implant is often used which is sometimes cage-shaped and is referred to as a spinal cage. The latter is fitted between the adjacent vertebrae. Such an implant will also be referred to below as a spinal cage. Such a cage ensures, on the one hand, that the space created by removal of the intervertebral disk is filled and the stability of the spinal column is thus maintained. On the other hand, the shape of spinal cages also often permits new bone to form in the area of the cage or in adjoining areas, thereby resulting in further stabilization.

Many different materials are already used for producing spinal cages. Because of its advantageous stability, titanium can be used, for example, in which case such an implant generally remains in the body. Moreover, various polymers are often used for this purpose, for example polylactides, polyglycolides or copolymers. Some of these materials have the advantage of being bioabsorbable, such that they are gradually permeated and replaced by endogenous material, in particular by bone substance. Furthermore, various ceramics are also used, for example those based on hydroxyapatite. Some of these materials too have the advantage of being degradable in the body.

A problem with materials used in conventional spinal cages is often that the material is either not degradable and remains in the body, or that in some cases a further operation has to be performed in order to remove the foreign body. Moreover, the degradation of the materials used within the body can often give rise to substances whose influence on endogenous functions is difficult to estimate. In some cases, undesirable side effects have to be expected. Starting out from this, the object of the invention is to make available a spinal cage which is made of bioabsorbable material and which, by being degraded within the body, does not cause any adverse side effects. In addition, a spinal cage of this type should have sufficient stability in order to avoid a collapse of the adjacent vertebrae. Moreover, the rate of degradation or the bioabsorption of the spinal cage in the body must be able to be controlled and adjusted in order to be able to load the affected segment of the spinal column in a manner adapted to the individual case and in order to take account of the other circumstances of the patient.

These objects are achieved by an implantable body as described in claim 1. Preferred embodiments of this implantable body are set forth in the dependent claims. The wording of all the claims is incorporated by reference into the content of the description.

The implantable body according to the invention, or spinal cage, for intersomatic fusion or for the above-described spinal fusion, is characterized in that it is made of a bioabsorbable, metallic material. The use of a metallic material has the advantage that this generally does not trigger any defensive reactions or rejection by the body. The bioabsorbability of the spinal cage, or its degradation by endogenous activity, means that the spinal cage is completely replaced by endogenous material, in particular by bone substance, and no exogenous material therefore has to remain in the patient. This also completely avoids the follow-up operations that are sometimes needed for removal of the implant or spinal cage.

A further advantage of the metallic materials used is that such materials have particularly favorable mechanical properties, particularly with regard to elasticity, deformability and stability, with low weight. This means that these materials can be used to produce suitable and advantageous spinal cages of different configurations and geometry. For example, the spinal cage can be designed as a solid body in the form of a disk or the like. However, it is particularly preferably designed as a hollow body, with the metallic materials used ensuring sufficient stability.

The spinal cage is particularly advantageously designed such that bone substance can grow through the spinal cage. This infiltration by bone means that initially, that is to say after the implantation, the stability of the vertebrae or of the spinal column is secured by the spinal cage. Later, after the osseous infiltration and in particular the bioabsorption of the spinal cage, this function is taken over by endogenous bone substance.

The metallic material, or its main component, can be in particular alkali metals, alkaline earth metals, iron, zinc or aluminum. The material is advantageously magnesium or iron. It is particularly advantageous if the material is an alloy or a sintered metal. The main component of the metallic material is particularly preferably magnesium or iron. Main component in this connection is to be understood as the component that makes up more than 50% of the particular material. All the percentages given in this connection relate to percent by weight. Examples of subsidiary components that can be used are manganese, cobalt, nickel, chromium, copper, cadmium, lead, tin, thorium, zirconium, silver, gold, palladium, platinum, rhenium, silicon, calcium, lithium, aluminum, zinc, carbon, sulfur, magnesium and/or iron.

It is particularly preferable if the material is a magnesium alloy containing up to 40% lithium and at least one iron addition. In another preferred embodiment of the invention, the metallic material can be an iron alloy advantageously containing a small proportion of aluminum, magnesium, nickel and/or zinc.

Preferred compositions of the metallic material can be as follows, for example:
50 to 98% magnesium
0 to 40% lithium
0 to 5% iron
0 to 5% other metals
55 to 65% magnesium
30 to 40% lithium
0 to 5% other metals
88 to 99% iron
0.1 to 4% chromium
0.1 to 3.5% nickel
0 to 5% other metals
90 to 96% iron
3 to 6% chromium
1 to 3% nickel
0 to 5% other metals

The in vivo degradation is generally effected by corrosion, and the latter can take place in a defined and foreseeable manner. The rate of degradation in the body can be influenced or controlled and predetermined by the composition of the metallic material. The rate of degradation of the spinal cage and its dwell time in the body can therefore be advantageously adjusted. In addition to the composition of the metallic material, the thickness of the material and the shape of the implant also play a role in defining the speed of corrosion and the speed of degradation. Depending on each particular case, in which the required support function of the implant generally has to be taken into consideration, the composition and the degradation of the spinal cage can be chosen such that the implant is degraded in the body within a few days or within several months. Since a build-up of bone usually takes place quite slowly, it is generally preferable for complete degradation of the spinal cage not to occur until after a few weeks or a few months.

The use of magnesium as component, in particular as main component, of the metallic material has the advantage that magnesium is physiologically very well tolerated. Moreover, particularly with magnesium alloys, a suitable choice of the other components of the alloy can be used to very precisely adjust the speed of degradation in the body. The use of iron as component, in particular as main component, of the metallic material has the advantage that iron alloys have excellent mechanical stability, which in many cases can be advantageous. With iron alloys of this kind, it is possible in particular to produce implants which have a very low wall thickness but which nevertheless ensure the required stability.

In a particularly preferred embodiment of the implantable body according to the invention, the body has a certain porosity. The pores are preferably micropores that have diameters in the range of from a few μm to mm, such as to permit incorporation of endogenous substance. The porosity allows endogenous cells, in particular bone-forming cells or cartilage-forming cells, to grow into the spinal cage, such that integration of the spinal cage can first take place, followed by its degradation advantageously in the body from the inside outward. The porosity of the implantable body thus, on the one hand, permits an improved integration and associated stabilization in the affected area of the spinal column. On the other hand, it also influences, in particular increases, the speed of degradation of the spinal cage, which may be preferable in certain circumstances.

The implantable body according to the invention can be a more or less solid body or a hollow body. A hollow body has the advantage that less exogenous material overall is introduced in the operation, and this may in some cases be advantageous for the process of degradation. However, a hollow body must be able to provide sufficient stability. This is achieved by a suitable choice of the shape and geometry, the wall thickness and the material used. The implantable body according to the invention can have, for example, an approximately disk-shaped form. However, an open structure is particularly preferred, for example in the form of a ring, a horseshoe, a cross or a star, the size of which is adapted to filling the intervertebral space. In another preferred embodiment, such an open structure, or even a closed structure, can be provided with other substructures, for example with in general surface recesses, openings, holes, cavities and/or slits. These different structures and substructures on the one hand facilitate the integration of the implant in the body and on the other hand promote the desired stability of the spinal column. Moreover, particularly by means of the substructures, the friction of the implant against the adjacent vertebrae is increased, such that the hold of the implant inside the spinal column is improved. In addition, the degradation rates can also be influenced, in particular accelerated, by this means. Moreover, such open structures and substructures reduce the amount of foreign material introduced by the spinal cage into the body, and this is generally advantageous for the reactions by the body. Material costs can also be reduced in this way.

As regards the design of the implantable body, the surfaces (cover plates) that adjoin the vertebral bodies after implantation can be oriented substantially parallel or at an angle to one another. The choice of shape depends mainly on the position that the spinal cage to be inserted assumes within the spinal column. The spinal column can in principle be divided into three areas. These are the cervical area (neck area), the thoracic area (chest area) and the lumbar area (lower back area). Substantially parallel cover plates are suitable especially for the cervical area, whereas in the lumbar area, where the spinal column generally has a greater curvature, an angled orientation of the cover plates is preferred.

In another preferred embodiment of the implantable body according to the invention, the body is provided with at least one active substance, in particular a biologically active substance. Growth factors, cytostatic agents, radioactive materials, antibiotics and/or antibodies are preferred in particular for this doping of the implantable body. The implantable body can even be protected by such substances, for example from bacterial decomposition before introduction into the body. Doping of this kind can also provide the implantable body with certain functions that have advantageous effects within the patient's body.

By using growth factors and osteoinductive factors, it is possible in particular to induce or promote the formation of new bone and/or new cartilage, as a result of which the integration and healing processes in the patient are accelerated. Examples of suitable growth factors are BMP (bone morphogenetic protein), in particular BMP-2 and/or BMP-4, and IGF (insulin-like growth factor), in particular IGF-I, and TGF (transforming growth factor), in particular TGF-βI. The implantable body can also be provided with one or more cytostatic agents, for example with cortisone. This is advantageous particularly in the case of cancerous changes to the vertebral bodies or the surrounding tissue. In this connection, it is also possible to use radioactive material, which is also suitable for destroying degenerated tissue, especially within local areas. Introduction of radioactive material into the patient by means of the implantable body may also be advantageous from the diagnostic point of view. Moreover, the use of antibiotics as doping agents in the implantable body is preferred. These can, on the one hand, prevent defense reactions in the body and can, on the other hand, help preserve the implantable bodies prior to the actual operation. In addition, the use of antibodies may also be of use in this connection, on the one hand for therapeutic reasons, and, on the other hand, for diagnostic reasons. These various substances, which have been mentioned as examples, and other active substances too, can be combined with one another and thus achieve particularly advantageous effects. Which doping substances are chosen will of course depend on each specific case.

The active substances can be applied in the form of a coating onto the implantable body. On the other hand, hollow cavities or internal spaces can also be provided with the substances, for example in the form of an internal coating or filler. It is particularly preferable if active substances are used in such a way that a controlled release is permitted. This can be achieved, for example, by the internal spaces of the implantable body being provided with the active substances, and by these internal spaces becoming exposed during the course of the bioabsorption of the implantable body, as a result of which the active substances are therefore only then released.

In a particularly preferred embodiment of the implantable body according to the invention, the body is provided with an extract of demineralized bone material (DBM). Such an extract contains various substances, in particular biologically active substances, which are very active in respect of formation of new bone and cartilage. As regards the components and the production of such an extract, reference is made to the disclosures of international patent applications WO 91/06324 and WO 93/20857. Such an extract is sold by the applicant under the brand name “Colloss”. Such an extract can preferably be used in combination with other active substances, in articular with other biologically active substances.

In another preferred embodiment of the implantable body according to the invention, this body is in a packaged form. It is particularly preferable if the body is packaged in a sterile state. The implantable body is dispatched and/or stored in such a package before use by the surgeon. During an operation, the implantable body can easily be removed from the particularly sterile package and implanted. Various materials are suitable as packaging material, for example plastic covers or the like. To ensure a sterile state, it is possible, for example, for the body to be irradiated before packaging, or also when inside the package, for example with radioactive rays. Other conventional sterilizing methods known to persons skilled in the art are also suitable.

Further features of the implantable body according to the invention will become clear from the following description of examples in combination with the dependent claims and the drawings. The various features here can be realized either singly or in combination with one another.

In the figures:

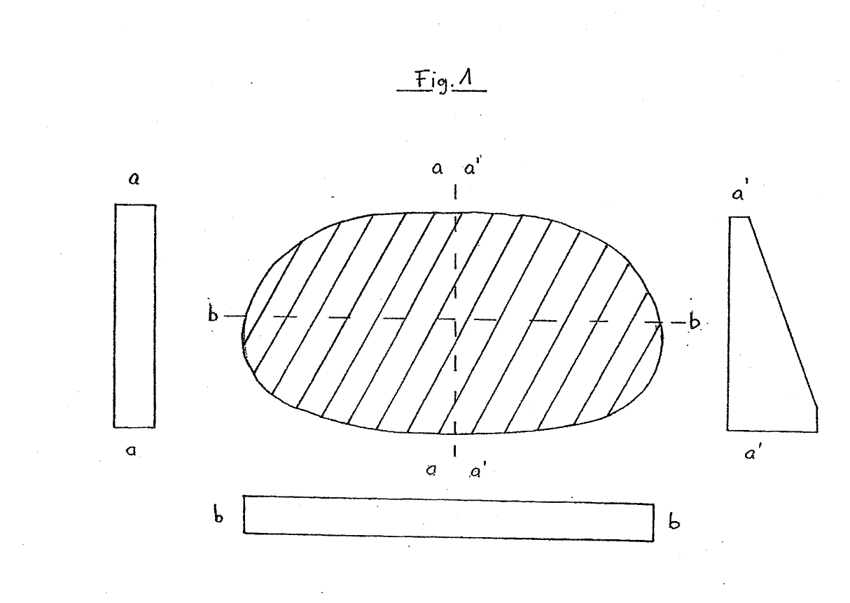

FIG. 1 shows an illustrative embodiment of the implantable body according to the invention;

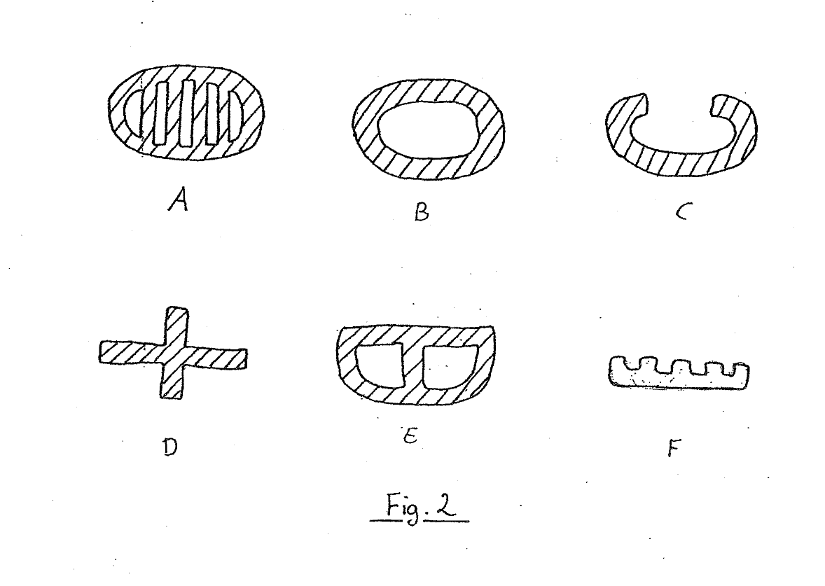

FIG. 2 shows various illustrative embodiments of the implantable body according to the invention in diagrammatic plan views (A-E), and in a cross section (F) through an implantable body according to the invention;

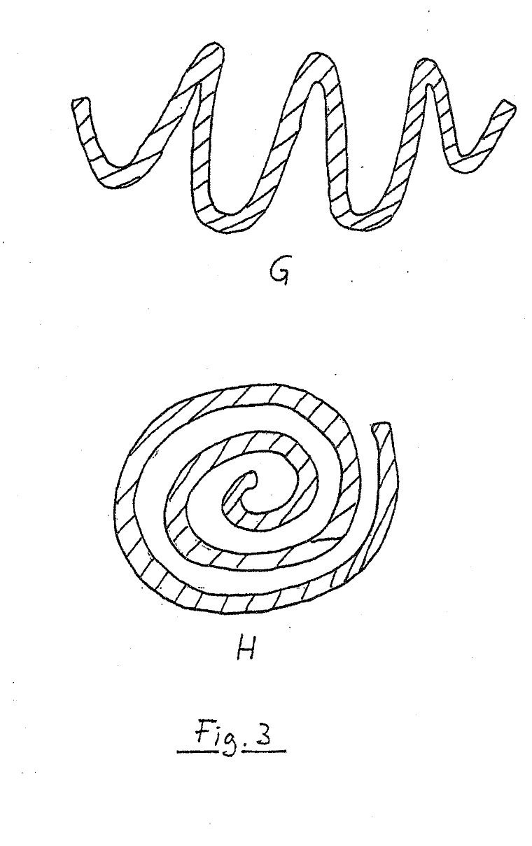

FIG. 3 shows two further illustrative embodiments of the implantable body according to the invention in diagrammatic plan views.

### EXAMPLES

FIG. 1 shows, by way of example, a plan view of a substantially disk-shaped implantable body which can be designed as a more or less solid body or as a hollow body. The surfaces of this body (cover faces) which adjoin the surrounding vertebral bodies after implantation can either extend substantially parallel to one another or at an angle to one another, as represented by sections a-a and a′-a′, respectively. The section b-b in the longitudinal direction shows the preferred parallel orientation of the cover faces in this direction.

FIG. 2 shows various other possible embodiments of the implantable body or spinal cage. The various forms are dimensioned in such a way that they largely correspond to the size of an intervertebral disk, so as to be able to fill the space that arises when a fusion operation is performed on the spinal column by removing the intervertebral disk material between two vertebrae. The configuration of the cover faces can be as shown in FIG. 1. FIG. 2A shows a plan view of an implantable body which has a plurality of more or less slit-shaped recesses. The amount of material to be introduced into the body is thus reduced, while at the same time this promotes integration and bioabsorption of the implant located in the patient. FIG. 2B shows a plan view of an annular implantable body, and FIG. 2C shows a plan view of an approximately horseshoe-shaped implantable body. FIG. 2D shows a plan view of an implantable body according to the invention which is designed in the shape of a cross. FIG. 2E shows a plan view of another possible embodiment of the implantable body. FIG. 2F shows a cross section through an implantable body which, on one face, has various surface recesses or cavities. This also means that the amount of material can be reduced and the integration of the implanted body can be accelerated and the fusion of the vertebrae achieved more quickly. Moreover, recesses of this kind are especially suitable for applying active substances, in particular biologically active substances such as growth factors or cytostatic agents, onto or into the implantable body, since the recesses afford an enlarged surface area. Moreover, the enlarged surface area forms stronger points of attack for bioabsorption, thereby accelerating the degradation of such an implant.

FIG. 3 shows two further possible embodiments of the implantable body. Spiral-shaped implantable bodies in various forms are also possible.

1. An implantable body for intersomatic fusion, characterized in that it is made from a bioabsorbable, metallic material.

2. The implantable body as claimed in claim 1, characterized in that the metallic material is magnesium or iron, or it contains magnesium or iron as its main component.

3. The implantable body as claimed in claim 1, characterized in that the material is a magnesium alloy.

4. The implantable body as claimed in claim 1, characterized in that the material is an iron alloy.

5. The implantable body as claimed in claim 1, characterized in that the body is porous, in particular microporous.

6. The implantable body as claimed in claim 1, characterized in that the body has an open structure and/or a structure with holes, cavities and/or slits.

7. The implantable body as claimed in claim 1, characterized in that the body is provided with at least one active substance, in particular a biologically active substance, preferably with at least one growth factor, a cytostatic agent, a radioactive material, an antibiotic and/or an antibody.

8. The implantable body as claimed in claim 1, characterized in that the body is provided with an extract of demineralized bone material.

9. The implantable body as claimed in claim 1, characterized in that the body is packaged, in particular in a sterile state.
